SEQUENCE LISTING (2 sequences)

SEQ ID NO: 1 - protein, 486 aa, Geobacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFQFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTT

SEQ ID NO: 2 - protein, 483 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRYQETSGEYNIQAWTGFNFPGRGTTYSNWKWQWFHFDGTDWDQSRSLSRIFKFHGKAWDWPVSSENGNYDYLMYADYDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGAYDMRNILNNTLVASNPTKAVTLVENHDTQPGQSLESTVQPWFKPLAYAFILTRSGGYPAVFYGDMYGTKGTTTYEIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

(19) United States
(12) Patent Application Publication
(10) Pub. No.: US 2023/0167386 A1
(43) Pub. Date: Jun. 1, 2023

(54) UNINHIBITED AMYLASES FOR BREWING WITH HIGH TANNIN MATERIALS

(71) Applicant: DUPONT NUTRITION BIOSCIENCES APS, Kongens Lyngby (DK)

(72) Inventor: Jacob Flyvholm Cramer, Højbjerg (DK)

(21) Appl. No.: 17/926,367

(22) PCT Filed: May 19, 2021
(86) PCT No.: PCT/US2021/033074

§ 371 Date: Nov. 18, 2022

Related U.S. Application Data
(60) Provisional application No. 63/028,042, filed on May 21, 2020.

(51) Int. Cl. C12C 5/00; C12N 9/28; C12C 7/04; C12C 11/00

(52) U.S. Cl. CPC C12C 5/004; C12C 7/04; C12C 11/003; C12N 9/2417; C12Y 302/01001

(57) ABSTRACT

The present invention provides methods of mashing high tannin adjuncts. More specifically, high tannin adjuncts can be refractory to exogenous enzymes used in barley malt brewing. The present invention provides enzymes which are uninhibited in tannin. In particular, tannin uninhibited, raw starch degrading α-amylases are provided in accordance with the instant invention.

### CROSS REFERENCE TO RELATED APPLICATIONS

This application is an international PCT application, which claims the benefit of U.S. Provisional Application No. 63/028,042, filed May 21, 2020, which is are hereby incorporated by reference in its entirety.

### TECHNICAL FIELD

The present invention relates to methods of mashing high tannin adjuncts. More specifically, the instant disclosure provides methods and compositions wherein a tannin uninhibited raw starch degrading α-amylase is employed in brewing to provide a wort from a high tannin adjunct.

### BACKGROUND OF THE INVENTION

Brewing generally involves three steps: malting, mashing and fermentation. The main purpose of the malting step is to develop enzymes which have a subsequent role during the brewing process in starch and protein degradation. Though traditionally beer has been brewed from just barley malt, hops and water; malt is an expensive raw material because it requires superior quality grains, water for germination and energy for kilning. To lower the cost of raw materials, unmalted grains, also called adjuncts, such as maize, rice, cassava, wheat, barley, rye, oat, quinoa and sorghum, maybe included in the brewing process. Adjuncts are primarily used because they are readily available and provide fermentable carbohydrates at a lower cost than barley malt.

The use of adjuncts in brewing complicates the traditional brewing process. Many enzymes have been developed for improving various aspect of beer production using malted barley as a starch source. However, it is known that high tannin adjuncts may not be readily processed by enzymes.

There is a continuing need for methods by which high tannin adjuncts can be used in beer production.

### SUMMARY OF THE INVENTION

In accordance with an aspect of the present invention, a method is presented for production of a Brewer’s wort having the step of mashing a grist having a high tannin adjunct in the presence of an exogenously supplied enzyme composition having a tannin uninhibited enzyme to provide the Brewer’s wort. Optionally, the tannin uninhibited enzyme is selected from the group consisting of a bacterial raw starch degrading α-amylase, glucoamylase, pullulanase, fungal alpha-amylase and a maltogenic alpha-amylase.

Optionally, the grist has sorghum. Optionally, the grist is at least 10% sorghum.

Optionally, the grist is at least 20% sorghum, at least 30% sorghum, at least 40% sorghum, at least 50% sorghum, at least 60% sorghum, at least 70% sorghum, at least 80% sorghum, at least 90% sorghum or 100% sorghum.

In another aspect of the invention, the grist optionally also has corn, cassava, barley, wheat, rye, millet or rice. In still other preferred embodiments, the grist optionally has at least 10 µM CAE/g of grist, at least 20 µM CAE/g of grist, at least 30 µM CAE/g of grist, at least 40 µM CAE/g of grist, at least 50 µM CAE/g of grist, at least 60 µM CAE/g of grist, at least 70 µM CAE/g of grist, at least 80 µM CAE/g of grist, at least 90 µM CAE/g of grist, at least 100 µM CAE/g of grist, at least 110 µM CAE/g of grist, at least 120 µM CAE/g of grist, at least 130 µM CAE/g of grist, at least 140 µM CAE/g of grist or at least 150 µM CAE/g of grist.

Optionally, the tannin uninhibited enzyme is a raw starch degrading α-amylase. Optionally, the raw starch degrading α-amylase is of class GH13. Optionally, the raw starch degrading α-amylase is derived from Cytophaga sp. Optionally, the raw starch degrading α-amylase has at least 60% sequence identity to SEQ ID NO:2 or an amylase active fragment thereof, at least 65% sequence identity to SEQ ID NO:2 or an amylase active fragment thereof, at least 70% sequence identity to SEQ ID NO:2 or an amylase active fragment thereof, at least 75% sequence identity to SEQ ID NO:2 or an amylase active fragment thereof, at least 80% sequence identity to SEQ ID NO:2 or an amylase active fragment thereof, at least 85% sequence identity to SEQ ID NO:2 or an amylase active fragment thereof, at least 90% sequence identity to SEQ ID NO:2 or an amylase active fragment thereof, at least 95% sequence identity to SEQ ID NO:2 or an amylase active fragment thereof, at least 98% sequence identity to SEQ ID NO:2 or an amylase active fragment thereof, at least 99% sequence identity to SEQ ID NO:2 or an amylase active fragment thereof, or has a sequence as set forth in SEQ ID NO:2 or an amylase active fragment thereof.

Optionally, the exogenously supplied enzyme composition having a tannin uninhibited raw starch degrading α-amylase also has one or more of a protease, a fungal α-amylase, a maltogenic α-amylase, a glucoamylase and a lipase.

In another aspect of the present invention, a method is presented in which a Brewer’s wort produced as described above with a tannin uninhibited enzyme is fermented to obtain an alcoholic beverage. Optionally, the alcoholic beverage is a beer.

In another aspect of the present invention, a wort is presented produced as described above with a tannin uninhibited enzyme. In another aspect of the present invention, a beer is presented which is produced from the wort described above.

In another aspect of the present invention, a method is presented to determine whether an enzyme is inhibited by tannin having the steps of incubating the enzyme in the presence of tannin and detecting cross-linking of the enzyme to tannin. Optionally, the tannin is catechin.

Optionally, the enzyme is a brewing enzyme. Optionally, the brewing enzyme is selected from the group consiting of a raw starch degrading α-amylase, a protease, a fungal α-amylase, a glucoamylase, a maltogenic α-amylase and a lipase. Optionally, the enzyme is a raw starch degrading α-amylase.

Optionally, the α-amylase is of class GH13. Optionally, the raw starch degrading α-amylase is derived from Cytophaga sp.

Optionally, the raw starch degrading α-amylase used in the method of detecting cross-linking has at least 60% sequence identity to SEQ ID NO:2 or an amylase active fragment thereof, at least 65% sequence identity to SEQ ID NO:2 or an amylase active fragment thereof, at least 70% sequence identity to SEQ ID NO:2 or an amylase active fragment thereof, at least 75% sequence identity to SEQ ID NO:2 or an amylase active fragment thereof, at least 80% sequence identity to SEQ ID NO:2 or an amylase active fragment thereof, at least 85% sequence identity to SEQ ID NO:2 or an amylase active fragment thereof, at least 90% sequence identity to SEQ ID NO:2 or an amylase active fragment thereof, at least 95% sequence identity to SEQ ID NO:2 or an amylase active fragment thereof, at least 98% sequence identity to SEQ ID NO:2 or an amylase active fragment thereof, at least 99% sequence identity to SEQ ID NO:2 or an amylase active fragment thereof or has a sequence as set forth in SEQ ID NO:2 or an amylase active fragment thereof. Optionally, the step of detecting cross-linking is measuring turbidity.

### BRIEF DESCRIPTION OF THE DRAWINGS

FIG. 1 shows liquefaction of red sorghum (6.0 g milled red sorghum was mixed with 23.0 g water) using the RVA method as described in example 3 by the alpha-amylases according to table 2: 1) GsAA, 2) CsAA and 3-5) Termamyl SCDS.

FIG. 2 shows optical density/turbidity (OD 600 nm) measured in MTP plates after incubation (30° C. for 30 min followed by 4° C. for 30 min) of Catechin with the three alpha-amylases respectively: GsAA, un-inhibited CsAA and Termamyl SCDS in various dilutions.

FIG. 3 shows Optical density/turbidity (OD 600 nm) measured in MTP plates after incubation (4° C. for 30 min, 60° C. for 30 min followed by 4° C. for 23 hrs) of Catechin with the three alpha-amylases respectively: GsAA, un-inhibited CsAA and Termamyl SCDS in various dilutions.

### BRIEF DESCRIPTION OF SEQ ID NOS

SEQ ID NO: 1 sets forth the mature amino acid sequence of the alpha amylase variant from Geobacillus stearothermophilus, GsAA.

SEQ ID NO: 2 sets forth the mature amino acid sequence of the alpha amylase variant from Cytophaga sp., CsAA.

### DEFINITIONS AND ABBREVIATIONS

Unless defined otherwise, all technical and scientific terms used herein have the same meaning as commonly understood by one of ordinary skill in the art to which this disclosure belongs. Singleton et al., DICTIONARY OF MICROBIOLOGY AND MOLECULAR BIOLOGY, 2^{nd} ed., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

A “variant” or “variants” refers to either polypeptides or nucleic acids. The term “variant” may be used interchangeably with the term “mutant”. Variants include insertions, substitutions, transversions, truncations, and/or inversions at one or more locations in the amino acid or nucleotide sequence, respectively. The phrases “variant polypeptide”, “polypeptide variant”, “polypeptide”, “variant” and “variant enzyme” mean a polypeptide/protein that has an amino acid sequence that either has or comprises a selected amino acid sequence of or is modified compared to the selected amino acid sequence, such as SEQ ID NO: 1, 2, 3, 4 or 5.

As used herein, a “homologous sequence” and “sequence identity” with regard to a nucleic acid or polypeptide sequence means having about at least 100%, at least 99%, at least 98%, at least 97%, at least 96%, at least 95%, at least 94%, at least 93%, at least 92%, at least 91%, at least 90%, at least 88%, at least 85%, at least 80%, at least 75%, at least 70%, at least 65%, at least 60%, at least 55%, at least 50%, or at least 45% sequence identity to a nucleic acid sequence or polypeptide sequence when optimally aligned for comparison, wherein the function of the candidate nucleic acid sequence or polypeptide sequence is essentially the same as the nucleic acid sequence or polypeptide sequence the candidate homologous sequence is being compared with. In some embodiments, homologous sequences have between at least about 85% and 100% sequence identity, while in other embodiments there is between about 90% and 100% sequence identity, and in other embodiments, there is at least about 95% and 100% sequence identity.

Homology is determined using standard techniques known in the art (see e.g., Smith and Waterman, Adv. Appl. Math. 2: 482 (1981); Needleman and Wunsch, J. Mol. Biol. 48: 443 (1970); Pearson and Lipman, Proc. Natl. Acad. Sci. USA 85: 2444 (1988); programs such as GAP, BESTHT, FASTA, and TFASTA in the Wisconsin Genetics Software Package (Genetics Computer Group, Madison, WI); and Devereux el al., Nucleic Acid Res., 12: 387-395 (1984)).

The “percent (%) nucleic acid sequence identity” or “percent (%) amino acid sequence identity” is defined as the percentage of nucleotide residues or amino acid residues in a candidate sequence that is identical with the nucleotide residues or amino acid residues of the starting sequence. The sequence identity can be measured over the entire length of the starting sequence

Homologous sequences are determined by known methods of sequence alignment. A commonly used alignment method is BLAST described by Altschul et al., (Altschul et al., J. Mol. Biol. 215: 403-410 (1990); and Karlin et al, Proc. Natl. Acad. Sci. USA 90: 5873-5787 (1993)). A particularly useful BLAST program is the WU-BLAST-2 program (see Altschul et al, Meth. Enzymol. 266: 460-480 (1996)). WU-BLAST-2 uses several search parameters, most of which are set to the default values. The adjustable parameters are set with the following values: overlap span =1, overlap fraction = 0.125, word threshold (T) = 11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Other methods find use in aligning sequences. One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (Feng and Doolittle, J. Mol. Evol. 35: 351-360

). The method is similar to that described by Higgins and Sharp (Higgins and Sharp, CABIOS 5: 151-153 (1989)). Useful PILEUP parameters including a default gap weight of 3.00, a default gap length weight of 0.10, and weighted end gaps. The term “optimal alignment” refers to the alignment giving the highest percent identity score.

As used herein, the term “malt beverage” includes such foam forming fermented malt beverages as full malted beer, ale, dry beer, near beer, light beer, low alcohol beer, low calorie beer, porter, bock beer, stout, malt liquor, non-alcoholic malt liquor and the like. The term “malt beverages” also includes alternative malt beverages such as fruit flavored malt beverages, e. g. , citrus flavored, such as lemon-, orange-, lime-, or berry-flavored malt beverages, liquor flavored malt beverages, e. g., vodka-, rum-, or tequila-flavored malt liquor, or coffee flavored malt beverages, such as caffeine-flavored malt liquor, and the like.

As used herein, the term “beer” traditionally refers to an alcoholic beverage derived from malt, which is derived from barley, and optionally adjuncts, such as cereal grains, and flavored with hops. Beer can be made from a variety of grains by essentially the same process. All grain starches are glucose homopolymers in which the glucose residues are linked by either alpha-1, 4- or alpha-1,6-bonds, with the former predominating. The process of making fermented malt beverages is commonly referred to as brewing. The principal raw materials used in making these beverages are water, hops and malt. In addition, adjuncts such as common corn grits, refined corn grits, brewer’s milled yeast, rice, sorghum, refined corn starch, barley, barley starch, dehusked barley, wheat, wheat starch, torrified cereal, cereal flakes, rye, oats, potato, tapioca, and syrups, such as corn syrup, sugar cane syrup, inverted sugar syrup, barley and/or wheat syrups, and the like may be used as a source of starch. The starch will eventually be converted into dextrins and fermentable sugars. For a number of reasons, the malt, which is produced principally from selected varieties of barley, has the greatest effect on the overall character and quality of the beer. First, the malt is the primary flavoring agent in beer. Second, the malt provides the major portion of the fermentable sugar. Third, the malt provides the proteins, which will contribute to the body and foam character of the beer. Fourth, the malt provides the necessary enzymatic activity during mashing.

As used herein, the term “tannin” refers to the class of naturally occurring polyphenolic biomolecules that are found in plants, including in sorghum. Tannin is composed of flavonoids which are 15 carbon compounds having two phenyl rings and a heterocyclic ring.

“Catechin” is a particular flavonoid found in tannin which has the chemical name (2R,3S)-2-(3,4-dihydroxyphenyl)-3,4-dihydro-2H-chromene-3,5,7-triol.

As used herein, the term “CAE” refers to catechin equivalent.

As used herein, the term “a tannin uninhibited enzyme” refers to an enzyme that retains substantial activity in the presence of or after exposure to tannin.

A centipoise is one hundredth of a poise, or one millipascal-second (mPa·s) in SI units (1 cP = 10⁻³ Pa·s = 1 mPa·s).

As used herein, the “process for making beer” is one that is well known in the art, but briefly, it involves five steps: (a) mashing and/or adjunct cooking (b) wort separation and extraction (c) boiling and hopping of wort (d) cooling, fermentation and storage, and (e) maturation, processing and packaging. In the first step, milled or crushed malt is mixed with water and held for a period of time under controlled temperatures to permit the enzymes present in the malt to convert the starch present in the malt into fermentable sugars. In the second step, the mash is transferred to a “lauter tun” or mash filter where the liquid is separated from the grain residue. This sweet liquid is called “wort” and the left over grain residue is called “spent grain”.

The mash is typically subjected to an extraction, which involves adding water to the mash in order to recover the residual soluble extract from the spent grain. In the third step, the wort is boiled vigorously. This sterilizes the wort and helps to develop the colour, flavour and odour. Hops are added at some point during the boiling. In the fourth step, the wort is cooled and transferred to a fermenter, which either contains the yeast or to which yeast is added. The yeast converts the sugars by fermentation into alcohol and carbon dioxide gas; at the end of fermentation the fermenter is chilled or the fermenter may be chilled to stop fermentation. The yeast flocculates and is removed. In the last step, the beer is cooled and stored for a period of time, during which the beer clarifies and its flavor develops, and any material that might impair the appearance, flavor and shelf life of the beer settles out. Prior to packaging, the beer is carbonated and, optionally, filtered and pasteurized. After fermentation, a beverage is obtained which usually contains from about 2% to about 10% alcohol by weight. The non-fermentable carbohydrates are not converted during fermentation and form the majority of the dissolved solids in the final beer. This residue remains because of the inability of malt amylases to hydrolyze the alpha-1,6-linkages of the starch. The non-fermentable carbohydrates contribute about 50 calories per 12 ounces of beer.

As used herein, the “process for making beer” may further be applied in the mashing of any grist.

As used herein, the term “grist” refers to any starch and/or sugar containing plant material derivable from any plant and plant part, including tubers, roots, stems, leaves and seeds. The grist may comprise grain, such as grain from barley, wheat, rye, oat, corn, rice, milo, millet and sorghum, and more preferably, at least 10%, or more preferably at least 15%, even more preferably at least 25%, or most preferably at least 35%, such as at least 50%, at least 75%, at least 90% or even 100% (w/w) of the grist of the wort is derived from grain. In some embodiments the grist may comprise the starch and/or sugar containing plant material obtained from cassava [Manihot esculenta] roots. The grist may comprise malted grain, such as barley malt. Preferably, at least 10%, or more preferably at least 15%, even more preferably at least 25%, or most preferably at least 35%, such as at least 50%, at least 75%, at least 90% or even 100% (w/w) of the grist of the wort is derived from malted grain.

The term “fermentation” means, in the context of brewing, the transformation of sugars in the wort, by enzymes in the brewing yeast, into ethanol and carbon dioxide with the formation of other fermentation by-products.

As used herein the term “malt” is understood as any malted cereal grain, such as barley.

The term “adjunct” is understood as the part of the grist which is not barley malt. The adjunct may be any carbohydrate rich material, e.g., sorghum, corn, cassava, wheat, rye, millet, rice, etc.

The term “mash” is understood as aqueous starch slurry, e. g. comprising crushed barley malt, crushed barley, and/or other adjunct or a combination hereof, mixed with water later to be separated into wort + spent grains.

As used herein, the term “wort” refers to the unfermented liquor run-off following extracting the grist during mashing.

As used herein, the term “spent grains” refers to the drained solids remaining when the grist has been extracted and the wort separated from the mash.

As used herein, the term “beer” refers to fermented wort, e.g. an alcoholic beverage brewed from barley malt, optionally adjunct and hops.

As used herein, the term “extract recovery” in the wort is defined as the sum of soluble substances extracted from the grist (malt and adjuncts) expressed in percentage based on dry matter.

As used herein, the term “pasteurization” means the killing of micro-organisms in aqueous solution by heating. Implementation of pasteurization in the brewing process is typically through the use of a flash pasteurizer or tunnel pasteurizer. As used herein, the term “pasteurization units or PU” refers to a quantitative measure of pasteurization. One pasteurization unit (1 PU) for beer is defined as a heat retention of one minute at 60° C. One calculates that:
PU = t x 1.393^(T - 60), where:
t = time, in minutes, at the pasteurization temperature in the pasteurizer
T = temperature, in degrees Celsius, in the pasteurizer
[^(T -60) represents the exponent of (T-60)]

Different minimum PU may be used depending on beer type, raw materials and microbial contamination, brewer and perceived effect on beer flavor. Typically, for beer pasteurization, 14 - 15 PU are required. Depending on the pasteurizing equipment, pasteurization temperatures are typically in the range of 64 - 72° C. with a pasteurization time calculated accordingly. Further information may be found in “Technology Brewing and Malting” by Wolfgang Kunze of the Research and Teaching Institute of Brewing, Berlin (VLB), 3rd completely updated edition, 2004, ISBN 3-921690-49-8.

As used herein, the term “DP1” (degree of polymerization 1) means glucose or fructose. “DP2” denotes maltose and/or isomaltose. “DP3” means maltotriose, panose and isopanose. “DP4/4+” means dextrin or maltooligosaccharides of a polymerization degree of 4 or higher which are unfermentable.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Before the exemplary embodiments are described in more detail, it is to be understood that this disclosure is not limited to particular embodiments described, as such may, of course, vary. Although any methods and materials similar or equivalent to those described herein can be used in the practice or testing of the present disclosure, exemplary methods, and materials are now described.

As used herein and in the appended claims, the singular forms “a”, “an”, and “the” include plural referents unless the context clearly dictates otherwise. Thus, for example, reference to “a gene” includes a plurality of such candidate agents and reference to “the cell” includes reference to one or more cells and equivalents thereof known to those skilled in the art, and so forth.

The publications discussed herein are provided solely for their disclosure prior to the filing date of the present application. Nothing herein is to be construed as an admission that the present disclosure is not entitled to antedate such publication by virtue of prior invention.

### Abbreviations

In the disclosure and experimental section which follows, the following abbreviations apply: GA (glucoamylase); CAE (catechin equivalent); wt % (weight percent); ⁰C (degrees Centigrade); rpm (revolutions per minute); H₂O (water); dH₂O (deionized water); aa (amino acid); AA (alpha amylase); kD (kilodaltons); g or gm (grams); µg (micrograms); mg (milligrams); µL (microliters); ml and mL (milliliters); mm (millimeters); µm (micrometer); M (molar); mM (millimolar); µM (micromolar); U (units); V (volts); MW (molecular weight); m (mass); sec(s) or s(s) (second/seconds); min(s) or m(s) (minute/minutes); hr(s) or h(s) (hour/hours); cP centipoise; dwb (dry-weight basis); (RVA) Rapid Visco Analyzer; ABS (Absorbance); Abs600 (Absorbance at 600 nm); OD (optical density); OD 600 (optical density at 600 nm); AU (absorbance unit); FAN (Free Alpha-Amino Nitrogen); PCR (Polymerase chain reaction); EtOH (ethanol); MTP (microtiter plate); EBC (European brewing convention); (OE) Original Extract; DP (degree of polymerisation); N (Normal) and ppm (parts per million).

### DETAILED DESCRIPTION OF THE INENTION

Traditional beer brewing employs only barley, hops and water. Barley is malted by steeping in water and heating. Malting begins the process of liberating the endogenous enzymes in barley which can break down starch. Malted barley is then mashed, completing the process of liberating the endogenous barley enzymes and converting starch into fermentable sugars to produce a wort. Fermentable sugars are then converted into ethanol by fermentation. Because barley can be expensive, brewers have sought to use less expensive starch sources in brewing. Such alternative starch sources are called adjuncts and include corn, cassava, wheat, rye, millet, sorghum and rice and mixtures thereof.

Barley is a temperate cereal which grows best in cooler climates. In tropical and sub-tropical regions, it is not practical to grow barley for brewing and the high cost of importing barley from more cooler climates is prohibitive. Crops such as maize, rice and sorghum are much easier to cultivate in such regions. Another consideration is the demands of the fuel ethanol market which can divert much of local maize production.

If very small amounts of adjunct are used in comparison to the amount of barley malt, it is possible that the endogenous barley malt enzymes are capable of breaking down the adjunct starch. But as brewers use higher amounts of adjunct compared to barely or no barley at all (i.e. 100% adjunct beer), endogenous barley malt enzymes are insufficient to break down the added starch of the adjunct into fermentable sugars.

While the procedures for brewing with barley have been well developed, adjunct brewing has proven to be more challenging, particular given cost concerns. For example, malted sorghum is low in endogenous enzymes to break down starch. Both liquefaction and saccharification of sorghum malts have been reported as problematic in the literature in terms of producing acceptable levels of fermentable sugars from the endogenous sorghum starch. In order to make starch susceptible to yeast for fermentation, it must be broken down into fermentable sugars, e.g., glucose, maltose and maltotriose. First, the granular structure of starch is broken down via a process called gelatinization. Water is added to the cereal or adjunct in question along with heating. When the gelatinization temperature is reached (which varies depending on the cereal being gelatinized), the starch granules become swollen and leak, increasing viscosity, and the granular structure is lost.

Gelatinization is followed by liquefaction. Once the starch is gelatinized, the starch is susceptible to cleavage by enzymes. Typically, in commercial brewing, exogenous enzymes are added during the mashing process to increase starch breakdown. For example, an endo-acting, raw starch degrading, α-amylase may be employed to convert starch to oligosaccharides and to lessen the viscosity of the gelatinized starch for the saccharification step. Other enzymes that may be employed in mashing include proteases, lipases, fungal α-amylases and maltogenic α-amylases. In the saccharification step, glucoamylases are employed to break the oligosaccharides down into fermentable sugars.

In accordance with an aspect of the present invention, however, it has been discovered that adjuncts or mixes of adjuncts that are high in tannin can be refractory to enzyme treatment. Tannins are a naturally occurring polyphenolic compounds found in various plants, seeds, bark and fruit skins. Of the cereals used as adjunct in brewing, sorghum is very high in tannin. While not being bound by any particular theory, applicant has discovered that brewing enzymes may be inhibited and/or inactivated by tannin or compounds in tannin. Tannin may lead to enzyme inactivation by reacting with the enzyme and/or by cross-linking enzymes together and consequently inactivating the enzyme. In accordance with an aspect of the present invention, it has been determined that a relatively higher percentage of proline residues contributes to enzyme inactivation by tannin with enzymes having a higher proline content tending to be inactivated by tannin more than enzymes with a lower content of proline. The reactivity of proline residues to tannin inactivation may be increased by the placement of the proline residues at the surface of the enzyme molecule, increasing they accessibility of tannin interaction.

In accordance with an aspect of the present invention, a method is presented for production of a Brewer’s wort having the step of mashing a grist having a high tannin adjunct in the presence of an exogenously supplied enzyme composition having a tannin uninhibited enzyme to provide the Brewer’s wort.

Tannin uninhibited enzymes may be identified in accordance with an aspect of the present invention by determining the amount of enzyme activity that remains after exposure to tannin. In this regard, standard assays may be used in accordance with the present invention by incorporating tannin, or more preferably a standard component of tannin such as catechin, into an assay and determining what impact on enzyme activity is made by tannin. In accordance with an aspect of the present invention, tannin uninhibited enzymes preferably retain at least 10% activity in tannin solutions, at least 20% activity in tannin solutions, at least 30% activity in tannin solutions, at least 40% activity in tannin solutions, at least 50% activity in tannin solutions, at least at least 60% activity in tannin solutions, at least 70%) activity in tannin solutions, at least 80% activity in tannin solutions, at least 90% activity in tannin solutions, at least 95% activity in tannin solutions, at least 99% activity in tannin solutions or 100% activity in tannin solutions.

According to an aspect of the present invention, the tannin solution preferably contains catechin. More preferably, the catechin is about 0.1 mg/ml, about 0.2 mg/ml, about 0.3 mg/ml, about 0.4 mg/ml, about 0.5 mg/ml, about 0.6 mg/ml, about 0.8 mg/ml, about 0.9 mg/ml, about 1 mg/ml, about 1.5 mg/ml, about 2 mg/ml, about 2.5 mg/ml and about 3 mg/ml. Still more preferably, the catechin is about 2 mg/ml and the enzyme retains about 90% activity.

In another aspect of the present invention, a tannin uninhibited enzyme retains substantial activity in the presence of grist containing tannin. In accordance with an aspect of the present invention, tannin uninhibited enzymes preferably retain at least 10% activity in grist containing tannin, at least 20% activity in grist containing tannin, at least 30% activity in grist containing tannin, at least 40% activity in grist containing tannin, at least 50% activity in grist containing tannin, at least at least 60% activity in grist containing tannin, at least 70% activity in grist containing tannin, at least 80% activity in grist containing tannin, at least 90% activity in grist containing tannin, at least 95% activity in grist containing tannin, at least 99% activity in grist containing tannin or 100% activity in grist containing tannin.

In accordance with an aspect of the present invention, the grist has at least 10 µM CAE/g of grist, at least 20 µM CAE/g of grist, at least 30 µM CAE/g of grist, at least 40 µM CAE/g of grist, at least 50 µM CAE/g of grist, at least 60 µM CAE/g of grist, at least 70 µM CAE/g of grist, at least 80 µM CAE/g of grist, at least 90 µM CAE/g of grist, at least 100 µM CAE/g of grist, at least 110 µM CAE/g of grist, at least 120 µM CAE/g of grist, at least 130 µM CAE/g of grist, at least 140 µM CAE/g of grist or at least 150 µM CAE/g of grist.

More preferably, the tannin uninhibited enzyme retains about 90% activity in a grist having 20 µM CAE/g of grist.

Preferably, the tannin uninhibited enzyme is selected from the group consiting of a raw starch degrading α-amylase, a protease, a fungal α-amylase, a glucoamylase, a maltogenic α-amylase and a lipase. Still more preferably, the enzyme is a raw starch degrading α-amylase.

Yet more preferably, the α-amylase is a glucohydrolase of class GH13. In still more preferred embodiments, the raw starch degrading α-amylase is derived from Cytophaga sp.

Still more preferably, the raw starch degrading α-amylase used in the method of detecting cross-linking has at least 60% sequence identity to SEQ ID NO:2 or an amylase active fragment thereof, at least 65% sequence identity to SEQ ID NO:2 or an amylase active fragment thereof, at least 70% sequence identity to SEQ ID NO:2 or an amylase active fragment thereof, at least 75% sequence identity to SEQ ID NO:2 or an amylase active fragment thereof, at least 80% sequence identity to SEQ ID NO:2 or an amylase active fragment thereof, at least 85% sequence identity to SEQ ID NO:2 or an amylase active fragment thereof, at least 90% sequence identity to SEQ ID NO:2 or an amylase active fragment thereof, at least 95% sequence identity to SEQ ID NO:2 or an amylase active fragment thereof, at least 98% sequence identity to SEQ ID NO:2 or an amylase active fragment thereof, at least 99% sequence identity to SEQ ID NO:2 or an amylase active fragment thereof or has a sequence as set forth in SEQ ID NO:2 or an amylase active fragment thereof.

Preferably, the grist has sorghum. More preferably, the grist is at least 10% sorghum.

More preferably, the grist is at least 20% sorghum, at least 30% sorghum, at least 40% sorghum, at least 50% sorghum, at least 60% sorghum, at least 70% sorghum, at least 80% sorghum, at least 90% sorghum or 100% sorghum.

In another aspect of the invention, the grist preferably also has corn, cassava, barley, wheat, rye, millet or rice.

In another aspect of the invention, the exogenously supplied enzyme composition having a tannin uninhibited raw starch degrading α-amylase also has one or more of a protease, a fungal α-amylase, a glucoamylase, a maltogenic α-amylase and a lipase.

In another aspect of the present invention, a method is presented in which a Brewer’s wort produced as described above with a tannin uninhibited enzyme is fermented to obtain an alcoholic beverage. Preferably, the alcoholic beverage is a beer.

In another aspect of the present invention, a wort is presented produced as described above with a tannin uninhibited enzyme. In another aspect of the present invention, a beer is presented which is produced from the wort described above.

In another aspect of the present invention, a method is presented to determine whether an enzyme is inhibited by tannin having the steps of incubating the enzyme in the presence of tannin and detecting cross-linking of the enzyme to tannin. In accordance with this aspect of the present invention, the need for doing different assays for each enzyme is avoided. Preferably, the tannin is catechin. Preferably, the step of detecting cross-linking is done by measuring turbidity.

Preferably, the enzyme is a brewing enzyme. More preferably, the brewing enzyme is selected from the group consiting of a raw starch degrading α-amylase, a protease, a fungal α-amylase, a maltogenic α-amylase and a lipase. Still more preferably, the enzyme is a raw starch degrading α-amylase.

Yet more preferably, the α-amylase is a glucohydrolase class GH13. In still more preferred embodiments, the raw starch degrading α-amylase is derived from Cytophaga sp.

Still more preferably, the raw starch degrading α-amylase used in the method of detecting cross-linking has at least 60% sequence identity to SEQ ID NO:2 or an amylase active fragment thereof, at least 65% sequence identity to SEQ ID NO:2 or an amylase active fragment thereof, at least 70% sequence identity to SEQ ID NO:2 or an amylase active fragment thereof, at least 75% sequence identity to SEQ ID NO:2 or an amylase active fragment thereof, at least 80% sequence identity to SEQ ID NO:2 or an amylase active fragment thereof, at least 85% sequence identity to SEQ ID NO:2 or an amylase active fragment thereof, at least 90% sequence identity to SEQ ID NO:2 or an amylase active fragment thereof, at least 95% sequence identity to SEQ ID NO:2 or an amylase active fragment thereof, at least 98% sequence identity to SEQ ID NO:2 or an amylase active fragment thereof, at least 99% sequence identity to SEQ ID NO:2 or an amylase active fragment thereof or has a sequence as set forth in SEQ ID NO:2 or an amylase active fragment thereof. Preferably, the step of detecting cross-linking is measuring turbidity.

Preferably, the tannin uninhibited enzyme is selected from the group consisting of a bacterial raw starch degrading α-amylase, glucoamylase, pullulanase, fungal alpha-amylase and a maltogenic alpha-amylase. Still more preferably, the tannin uninhibited enzyme is a bacterial raw starch degrading α-amylase.

Preferably, the tannin uninhibited enzyme has a specific catechin cross-linking activity of less than 10 AU/µg, of less than 9 AU/µg, of less than 8 AU/µg, of less than 7 AU/µg, of less than 6 AU/µg, of less than 5 AU/µg, of less than 4 AU/µg, of less than 3 AU/µg, of less than 2 AU/µg or of less than 1 AU/µg.

### EXAMPLES

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### Example 1 - Enzymes

GsAA: An alpha amylase variant from Geobacillus stearothermophilus having the amino acid sequence shown in SEQ ID NO: 1.

CsAA: A tannin un-inhibited alpha amylase variant from Cytophaga sp. having the amino acid sequence shown in SEQ ID NO:2.

As an example of a liquifying alpha-amylase used in brewing, Termamyl SCDS from Novozymes, was used.

### Example 2 - Protein Determination Methods

### Protein Determination by Stain Free Imager Criterion

Protein was quantified by SDS-PAGE gel and densitometry using Gel Doc™ EZ imaging system. Reagents used in the assay: Concentrated (2x) Laemmli Sample Buffer (Bio-Rad, Catalogue #161-0737); 26-well XT 4-12% Bis-Tris Gel (Bio-Rad, Catalogue #345-0125); protein markers “Precision Plus Protein Standards” (Bio-Rad, Catalogue #161- 0363); protein standard BSA (Thermo Scientific, Catalogue #23208) and SimplyBlue Safestain (Invitrogen, Catalogue #LC 6060. The assay was carried out as follow: In a 96well-PCR plate 50 µL diluted enzyme sample were mixed with 50 µL sample buffer containing 2.7 mg DTT. The plate was sealed by Microseal ‘B’ Film from Bio-Rad and was placed into PCR machine to be heated to 70° C. for 10 minutes. After that the chamber was filled by running buffer, gel cassette was set. Then 10 µL of each sample and standard (0.125-1.00 mg/mL BSA) was loaded on the gel and 5 µL of the markers were loaded. After that the electrophoresis was run at 200 V for 45 min. Following electrophoresis, the gel was rinsed 3 times 5 min in water, then stained in Safestain overnight and finally destained in water. Then the gel was transferred to Imager. Image Lab software was used for calculation of intensity of each band. A calibration curve was made using BSA (Thermo Scientific, Catalogue #23208) and the amount of the target protein was determined by the band intensity and calibration curve. The protein quantification method was employed to prepare enzyme samples of used in subsequent Examples.

### Example 3 - Viscosity Analysis of Raw Material Upon Starch Gelatinization by RVA

The viscosity was analyzed by an RVA method based on AACC analysis (American Association of Cereal Chemists). The Rapid Visco Analyser (RVA) is a rotational viscometer able to continuously record the viscosity of a sample under conditions of controlled temperature. The ability of the RVA to suspend samples in a solvent, maintain them in suspension throughout the test, and apply an appropriate degree of shear to match processing conditions makes it particularly valuable in many process and research applications. An RVA from Newport Scientific (enabling viscosities between 0 cP and 24.000 cP) was utilized and controlled with Thermocline for Windows (TCW3) software program, that able heating and cooling of samples in the range 0-100° C. in steps of 0. 1° C. The RVA was equipped with a water bath from Thermo Scientific Accel 500 LC for temperature control. Initialization and auto-zero of the instrument were performed initially before each experiment at 960 rpm of the propeller, for warming up the motor, the oil on the RVA and ensure consistent measurements.

All raw material samples were prepared in Perten aluminum cans. 6.0 g raw material was mixed with 24.0 g preheated tap water (50° C.). pH was adjusted to 5.6 with acid or sodium hydroxide (2.5 M H₂SO₄ / 1 N NaOH). This resulted in a water to grist ratio of 4:1. The enzyme solution applied to the mix was 1.0 ml replacing 1.0 ml of the tap water in the sample. A rubber plug was placed on top of the small can and shaken for about 10 sec. to avoid lumps, special for flour and fine powders.

The chamber in the RVA was preheated to 50° C. and the following program for analysis of said starch liquefaction was used as given in the table 1 below:

TABLE 1

RVA Temperature (°C), time (hh:mm:ss) and speed (rpm) settings

| Time | Type | Value | Unit |
|---|---|---|---|
| 0:00:00 | Temp | 50 | °C |
| 0:00:00 | Speed | 500 | Rpm |
| 0:00:10 | Speed | 160 | Rpm |
| 0:01:00 | Temp | 50 | °C |
| 0:23:30 | Temp | 95 | °C |
| 0:23:30 | End | 95 | °C |

At end of analysis the RVA was cooled down to 50° C. to get ready for the next sample to be analyzed. The following characteristic were measured of each analysis: Pasting temperature of said starch, Peak viscosity, Peak time, Peak temperature and the Final viscosity.

### Example 4 - Viscosity Analysis of High Tannin Rich Red Sorghum Upon Starch Gelatinization with Alpha-Amylases

The viscosity was analyzed according to the RVA method described in example 3 of milled Red sorghum (Diago, Kenya, 27.11.2018, milled at a Buhler Miag malt mill 1.6 mm setting) with a high content of various tannins (> 1 g catechin equiv./100 g dwb). Thus 6.0 g milled Red sorghum was mixed with 23.0 g of preheated tap water (50° C.). The solution was mixed for about 10 sec. in the small beaker using a spatula and pH adjusted to 5.6 with 2.5 M H₂SO₄. 1 mL enzyme was added resulting in the given concentration show in table 2 below. Following the sample was mixed and a spindle was placed in the sample and start the measurement using the RVA.

TABLE 2

RVA samples and dosing of liquifying alpha-amylases

| No. | Raw Material | GsAA | CsAA | Termamyl SCDS |
|---|---|---|---|---|
| | | ppm on DS | ppm on DS | ppm on DS |
| 1 | Red Sorghum | 27 | | |
| 2 | Red Sorghum | | 18 | |
| 3 | Red Sorghum | | | 11 |
| 4 | Red Sorghum | | | 22 |
| 5 | Red Sorghum | | | 33 |

The RVA analysis of the given samples are shown in table 3. Surprisingly, the CsAA was the only alpha-amylase delivering complete viscosity reduction of the Sorghum sample. The high enzyme activity was also seen by clear lowering of the peak viscosity, reduction of peak time and a complete reduction of the final viscosity of 60 Cp by 18 ppm on DS un-inhibited CsAA, as compared to 940 Cp achieved by 27 ppm on DS GsAA and 5080 to 7274 Cp achieved by 11 to 33 ppm on DS dose of Termamyl SCDS. In addition, the very efficient degradation was also seen by reduced peak temperature, time and viscosity as compared to higher dosages of GsAA and Termamyl SCDS.

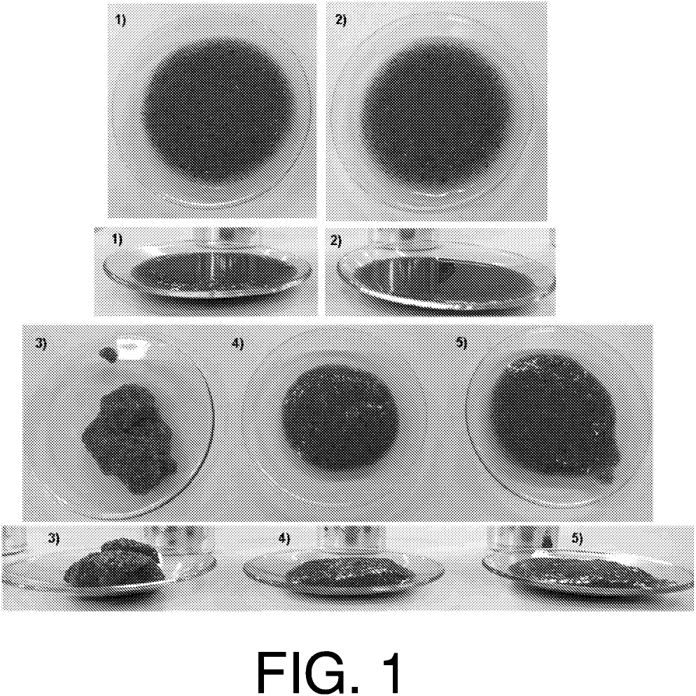

A photograph of the processed material is shown in FIG. 1, immediately after RVA processing. Here it is clearly seen that sample 2 processed by the un-inhibited CsAA alpha-amylase is much further solubilized with fewer solid particles seen as compared with Termamyl SCDS and minor extend GsAA.

TABLE 3

RVA analysis: Pasting temperature of said starch, Peak viscosity, Peak time, Peak temperature and Final viscosity

| Sample no. Running row | Pasting Temp | Peak Visc | Peak Time | Peak Temp | Final Visc |
|---|---|---|---|---|---|
| | °C | Cp | Minutes | °C | Cp |
| 1) | 72.1 | 7881 | 19.3 | 86.6 | 940 |
| 2) | 72.05 | 7274 | 17.9 | 83.6 | 60 |
| 3) | 72.1 | 10228 | 21.0 | 89.75 | 7274 |
| 4) | 72.05 | 9810 | 20.5 | 88.8 | 6078 |
| 5) | 72.1 | 9181 | 20.6 | 89.15 | 5080 |

### Example 5 - Infusion Mashing With Red Sorghum Using Un-Inhibited Alpha-Amylase

The objective of this example is to demonstrate the benefit of having an un-inhibited alpha-amylase present during processing of adjunct with high tannin content in an infusion process. Enzymes was tested in a mashing operation model system for wort production using milled Red and White sorghum (Diago, Kenya, 27.11.2018 and DK18-00735, milled at a Buhler Miag malt mill 1.6 mm setting) with a high content of various tannins.

### Mashing Operation for Wort Production

Sorghum grist (35.0 g milled White sorghum and 35.0 g milled Red sorghum) was mixed in beakers and mixed with 175 g of tap water in mashing bath (Lockner, LG-electronics) cups and pH adjusted to pH 5.4 with 2.5 M sulphuric acid., resulting in a water to grist ratio of 2.5:1. The alpha-amylases were added based on mg protein determined according to example 1 in the following setup: Trial 1, 13.4 µg GsAA per g sorghum; Trial 2, 20.2 µg GsAA per g sorghum; Trial 3, 17.7 µg un-inhibited CsAA per g sorghum and 26.5 µg un-inhibited CsAA per g sorghum. In addition to enable filterability, fermentable sugars and appropriate FAN levels the following enzymes were added fixed to each trial: 0.250 mg LAMINEX® BG2 (Dupont), 0.500 mg Alphalase® NP (Dupont), 0.500 mg DIAZYME® X4 (Dupont), 3.000 mg DIAZYME® MA (Dupont) and 1.000 mg DIAZYME® P10 (Dupont) all per g sorghum grist. The adjunct was mashed with the program; heated to 60° C. and kept for 30 minutes for mashing in; heated to 70° C. for 10.0 minutes by increasing temperature with 1° C./minute; kept at 70° C. for 45 minutes; heated to 75° C. for 5 minutes by increasing temperature with 1° C./minute; kept at 75° C. for 45 minutes; heated to 82° C. for 7 minute by increasing temperature with 1° C./minute; kept at 82° C. for 20 minutes and mashing off.

Hereafter, iodine negative was tested when temperature had reached 82° C. and mashed off. The time in minutes that was required to get iodine negative was noted and result are given in table 4. It here clear that the un-inhibited CsAA by a lower dosage and in shorter time were able to obtain an iodine negative result of the mash as compared to GsAA, indicating a further processed starch fraction of the sorghum material.

TABLE 4

Iodine testing of Sorghum mashing. The time in minutes that was required to get iodine negative is noted by OK for trial 1-4 with the following alpha-amylase addition: Trial 1, 13.4 µg GsAA per g sorghum; Trial 2, 20.2 µg GsAA per g sorghum; Trial 3, 17.7 µg un-inhibited CsAA per g sorghum and Trial 4, 26.5 µg un-inhibited CsAA per g sorghum.

| Trial | Iodine negative at 82° C. | | | | | |
|---|---|---|---|---|---|---|
| | 0 min | 5 min | 10 min | 15 min | 20 min | 25 min |
| 1 | | | | | OK- a bit dark | |
| 2 | | | | OK | | |
| 3 | | OK | | | | |
| 4 | OK | | | | | |

At the end of mashing, the mashes were cooled, made up to 350 g and filtered. Filtrate volumes were measured after 30 minutes. The pH was adjusted to pH 5.2 with 2.5 M sulphuric acid and one pellet of bitter hops from Hopfenveredlung, St. Johann: Alpha content of 16.0 % (EBC 7.7 0 specific HPLC analysis, 01.10.2013), was added to each flask (350 g). The wort samples were boiled for 60 minutes in a boiling bath and wort were cooled down to 17° C. and filtered and used for analysis, see below.

Wort analysis: Original Extract (OE), Extract in the wort samples after mashing was measured using Anton Paar (Lovis) following Standard Instruction Brewing, 23.8580-B28. FAN The content of Free Alpha-Amino Nitrogen (mg/litre) was measured in the wort following Standard Instruction Brewing, 23.8580-B15 using Spectrophotometer Genesys 10S UV-Vis (Based on EBC 8.10). Fermentable sugars (% total + g/100 ml) by HPLC were DP1, DP2, DP3 and DP4+ was determined after mashing following Standard Instruction Brewing, 23.8580-B20. Sugar wort composition was determined at a HPLC-RI system equipped with an RSO oligosaccharide column, Ag+ 4% crosslinked (Phenomenex, The Netherlands) and an analytical guard column (Carbo-Ag+ neutral, AJ0-4491, Phenomenex, The Netherlands) operated at 70° C. Isocratic flow of 0.3 ml/min was maintained throughout analysis with a total run time of 45 min and injection volume was set to 10 µL. Quantification was made by the peak area relative to the peak area of the given standard (DP1: glucose; DP2: maltose; DP3: maltotriose and peaks with a degree of four or higher maltotetraose was used as standard).

The result of HPLC and extract analysis of the wort after saccharification are shown in table 5 including the relative sugar composition in the wort is shown in table 5. It’s seen from data in table 5 that un-inhibited CsAA as compared to GsAA in both high and low dose delivered more fermentable sugars, as the sum of DP1-DP3 (avr. 72.5% vs avr. 70.8%) and more extract (13.84-13.90°P vs. 13.49-13.60°P). The un-inhibited CsAA is superior in liquifying and enabling saccharification of the said starch in the high tannin material.

TABLE 5

HPLC and original extract analysis of wort composition using different alpha-amylases.

| | Extract | HPLC - Relative sugar distribution | | | |
|---|---|---|---|---|---|
| | (°P) OE | DP1 % | DP2 % | DP3 % | DP4+ % |
| Trial 1, 13.4 µg GsAA per g sorghum; | 13.49 | 24.0 | 35.6 | 10.8 | 29.5 |
| Trial 2, 20.2 µg GsAA per g sorghum; | 13.60 | 24.5 | 35.8 | 10.9 | 28.9 |
| Trial 3, 17.7 µg un-inhibited CsAA per g sorghum | 13.84 | 26.3 | 33.8 | 12.5 | 27.3 |
| Trial 4, 26.5 µg un-inhibited CsAA per g sorghum | 13.90 | 26.2 | 32.9 | 13.1 | 27.7 |

The quantified Free Alpha-Amino Nitrogen (mg/litre) was measured in the wort and is shown in table 6. There was no difference in the FAN levels measured between trial 1-4 and variation was within the experimental error.

TABLE 6

FAN Free Alpha-Amino Nitrogen (mg/litre) measured of sorghum wort using different alpha-amylases in infusion mash.

| | Average FAN mg/litre |
|---|---|
| Trial 1, Low dose GsAA | 61 |
| Trial 2, High dose GsAA | 60 |
| Trial 3, 1 Low dose CsAA | 60 |
| Trial 4, High dose CsAA | 61 |

### Example 6 - Active Tannin Cross-Linking Activity With Alpha-Amylases

The objective of this example is to demonstrate the preference of various alpha-amylases to react and cross-link with reactive tannin species and hereby get inactivated. High polyphenol content as found in various sorghum species has been associated with impaired nutritional quality of the grain and with reduced brewing value. In this example we tested the cross-linking of one of the most reactive phenolic compounds in the tannin fraction, catechin, with various exogenously supplemented alpha-amylases.

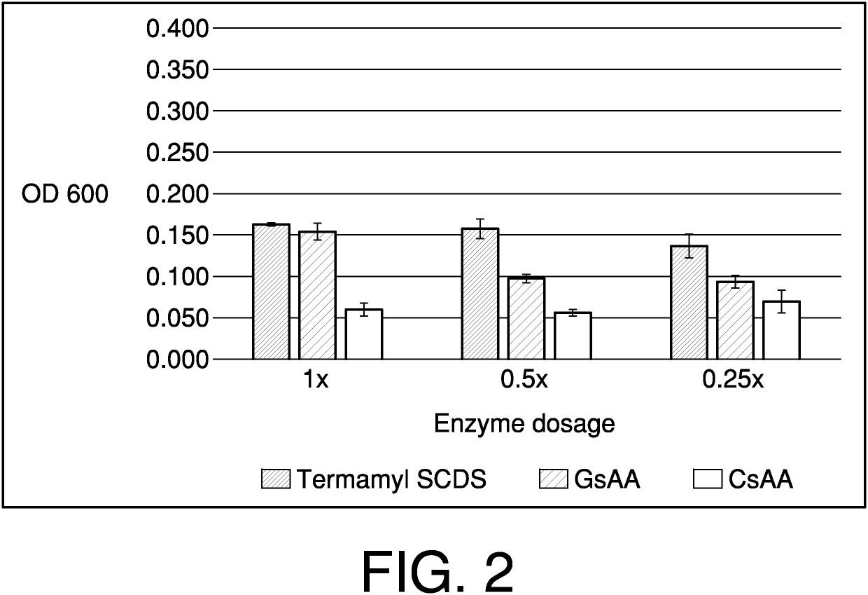

A Catechin solution (Sigma Aldrich C1251) was created by wetting the material with 70% ethanol and afterwards added to 20 mM Na-Phosphate/Acetate pH 4.5, 0.2%(v/v) ethanol to make a final concentration of 2 mg/ml. The enzymes GsAA, un-inhibited CsAA, Termamyl SCDS were all diluted in 20 mM Na-Phosphate/Acetate pH 4.5. The alpha-amylase concentration was 5.4 mg/g GsAA, 7.1 mg/g un-inhibited CsAA and 4.4 mg/g Termamyl SCDS, as determined according to example 2. 125 µL of the enzyme was mix with 125 µL 20 mM Na-Phosphate/Acetate pH 4.5 and 60 µL Catechin solution in a 96 well MTP plates (Corning, NY, USA) sealed with tape and incubated at 30° C. for 30 min followed by 4° C. for 30 min to promote cross-linking reaction between enzyme and poly-phenol. The amount of cross-linking was quantified as turbidity read by OD at 600 nm at a plate reader. The blank experiment was replacing buffer instead of enzyme. The resulting cross-linking or haze/turbidity formed are shown in FIG. 2 for three enzyme concentrations. All enzymes in the tested dilution showed increased cross-linking as compared to the control (buffer) resulting in an OD600 of 0.004 and higher responses with increased enzyme concentration. Noteworthy did un-inhibited CsAA show the lowest OD as compared to GsAA and Termamyl SCDS in all the tested dilutions, clearly demonstration lower cross-linking tendency or reactivity with catechin and hereby less inhibition. As the cross-linking clearly is enhanced by increased enzyme concentration, the finding of un-inhibited CsAA showing lowest cross-linking being applied with highest enzymes concentration significant underscores the low reactivity of CsAA with catechin.

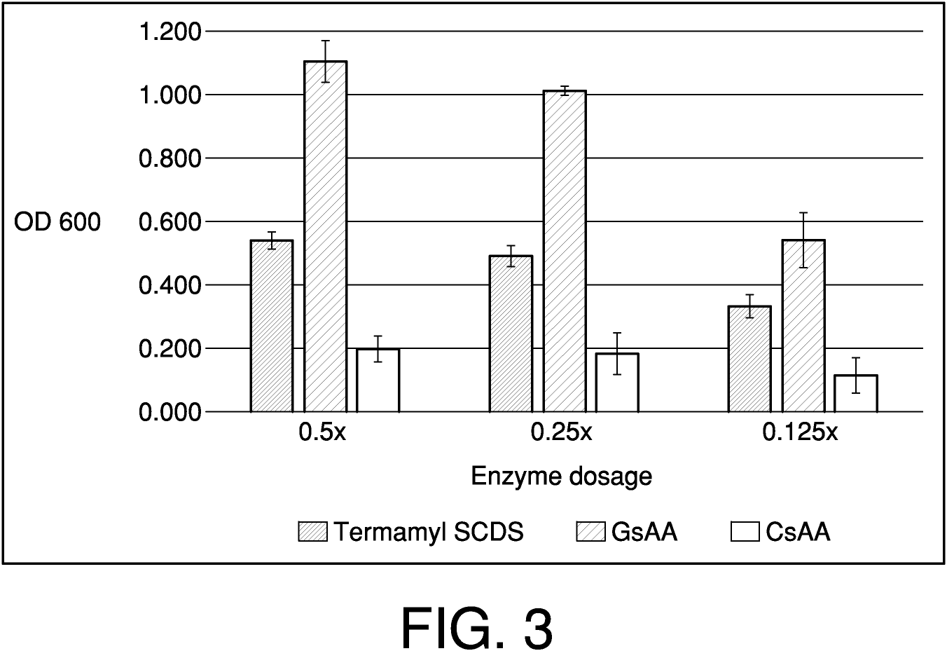

An additional cross-linking reaction was carried out with prolonged incubation time to enhance the turbidity generated. Similarly, the enzymes GsAA, un-inhibited CsAA, Termamyl SCDS were all diluted in 20 mM Na-Phosphate/Acetate pH 4.5 and the alpha-amylase concentration were 5.4 mg/g GsAA, 7.1 mg/g un-inhibited CsAA and 4.4 mg/g Termamyl SCDS, as determined according to example 2. Reactions were mixed as described above and sealed plates were incubated 30 min 4° C., 30 min 60° C. and 23 hrs 4° C. The results are shown in FIG. 3. The combination of increase temperature 60° C. for 30 min and prolonged incubation at 4° C. clearly increased catechin reactivity also at lowered enzyme dosages as compared with incubation 30° C. for 30 min followed by 4° C. for 30 min. Despite the increased reactivity, it was clear that CsAA showed the lowest OD (cross-linking) as compared to GsAA and Termamyl SCDS in all the tested dilutions. Furthermore, the prolonged catechin experiments underscores the low reactivity of CsAA with catechin and low inhibition. In this regard, one may calculate a relative specific cross-linking activity for each of the enzymes using the applied conditions (30 min 4° C., 30 min 60° C. and 23 hrs 4° C.) and an enzyme concentration in the assay between 0.4 to 0.8 mg/ml by the following:

$$C a t e c h i n c r o s s - l i n k i n g a c t i v i t y = \frac{A b s 600 \left(e n z y m e\right) - A b s 600 \left(n o e n z y m e\right)}{{m}_{e n z y m e}} ,$$

With Abs600(enzyme), being the absorbance measured in MTP at 600 nm after cross-linking reaction between catechin and enzyme, Abs600(no enzyme) being the absorbance measured in MTP at 600 nm after cross-linking reaction between catechin and water instead of enzyme and m_{enzyme} being the mass of the enzyme applied in the given assay. The specific cross-linking activity for the three enzymes was calculated in AU (absorbance unit)/µg and shown in table 7.

TABLE 7

Specific Catechin cross-linking activity AU (absorbance unit)/µg of three alpha-amylases.

| | AU/µg |
|---|---|
| Termamyl SCDC | 5.3 |
| GsAA | 8.9 |
| CsAA | 1.2 |

It can be seen that, CsAA has a very low specific catechin cross-linking activity of 1.2 AU/µg as compared to GsAA (8.9 AU/µg) and Termamyl SCDS (5.3 AU/µg).

### Example 7 - Amino Acid Composition of Alpha-Amylases

The content of proline residues in proteins has previously been positive correlated with polyphenol interaction and the mole percent of proline in a protein or polypeptide was shown to be essentially linearly related with the ability of that protein to form haze or precipitation with catechin (Asano et al., J.Am.Soc.Brew.Chem.,1982), (Siebert et al., Agric. Food Chem., 1996). The mole percent of proline in the polypeptide the studied alpha amylase variant where therefore analyzed.

The amino acid composition of the alpha amylase variant from Geobacillus stearothermophilus, GsAA, as set forth in SEQ ID NO: 1 is given below. Amino acid composition of SEQ ID NO: 1:

| | |
|---|---|
| Ala (A) 31 | 6.4% |
| Arg (R) 7 | 3.5% |
| Asn (N) 23 | 4.7% |
| Asp (D) 40 | 8.2% |
| Cys (C) 1 | 0.2% |
| Gln (Q) 18 | 3.7% |
| Glu (E) 17 | 3.5% |
| Gly (G) 46 | 9.5% |
| His (H) 11 | 2.3% |

| | |
|---|---|
| Ile (I) 19 | 3.9% |
| Leu (L) 32 | 6.6% |
| Lys (K) 30 | 6.2% |
| Met (M) 10 | 2.1% |
| Phe (F) 23 | 4.7% |
| Pro (P) 21 | 4.3% |
| Ser (S) 26 | 5.3% |
| Thr (T) 38 | 7.8% |
| Trp (W) 20 | 4.1% |
| Tyr (Y) 33 | 6.8% |
| Val (V) 30 | 6.2% |

The amino acid composition of the alpha amylase variant from Cytophaga sp., CsAA as set forth in SEQ ID NO: 2 is given below. Amino acid composition of SEQ ID NO: 2:

| | |
|---|---|
| Ala (A) 36 | 7.5% |
| Arg (R) 16 | 3.3% |
| Asn (N) 33 | 6.8% |
| Asp (D) 34 | 7.0% |
| Cys (C) 0 | 0.0% |
| Gln (Q) 18 | 3.7% |
| Glu (E) 18 | 3.7% |
| Gly (G) 49 | 10.1% |
| His (H) 8 | 1.7% |
| Ile (I) 15 | 3.1% |
| Leu (L) 27 | 5.6% |

| | |
|---|---|
| Lys (K) 27 | 5.6% |
| Met (M) 9 | 1.9% |
| Phe (F) 17 | 3.5% |
| Pro (P) 20 | 4.1% |
| Ser (S) 28 | 5.8% |
| Thr (T) 40 | 8.3% |
| Trp (W) 8 | 3.7% |
| Tyr (Y) 37 | 7.7% |
| Val (V) 33 | 6.8% |

It can be observed that the uninhibited CsAA has low mole percent of proline in the amylase polypeptide of 4.1% whereas the GsAA showing higher cross-linking reactivity and polyphenol inhibition has a noteworthy higher mole percent of proline in the amylase polypeptide of 4.3%.

### Example 8 - Total Flavonoid Content of Grains Used in Brewing

Total flavonoid content was assayed according to the modified versions of the method described by Shao et al. J Agr Food Chem. 2014 and expressed or calculated as micromole of catechin equivalent (CAE) per g of grain flour (µmol CAE/g). The determined flavonoid content of grains used in brewing is shown in table 8 below.

TABLE 8

Total calculated Flavonoid contents of grains were expressed as micromoles of catechin equivalent per gram of grain

| | |
|---|---|
| Grain | Total flavonoid concentration |
| Rice, General Mills, Golden Valley: | 0.60 ± 0.04 µmol/g of grain¹ |
| Oats, General Mills, Whole oats: | 0.71 ± 0.05 µmol/g of grain¹ |
| Barley, Dutch Barley: | 1.0 µmol/g of grain³ |
| Wheat, General Mills, Golden Valley: | 1.15 ± 0.03 µmol/g of grain¹ |
| Corn, Sunlite whole yellow corn: | 1.52 ± 0.17 µmol/g of grain¹ |
| Malt, Raw Barley malt: | 2.4 µmol/g of grain⁴ |
| Malt, Dark roasted Barley malt: | 7.57 µmol/g of grain⁴ |
| White Sorghum (type II) Feterita: | 15.8 µmol/g of grain² |
| White Sorghum (type II) White Tannin | 35.8 µmol/g of grain² |
| Red Sorghum Red tannin (type III) NS | 131.6 µmol/g of grain² |
| Red Sorhum PAN 3860 (type III) | 156.0 µmol/g of grain² |

¹ Liu et al., J. Agric. Food Chem. 2002
² Taylor et al., J. Inst. Brew. 2013, utilizing a catechin mw of 290.36 g/mol
³ Jende-Strid et al., Carlsberg Res. Commun., 1985
⁴ Xiang Ma et al., Food Funct., 2019, utilizing a catechin mw of 290.36 g/mol

What is claimed is:

1. A method for production of a Brewer’s wort comprising mashing a grist comprising a high tannin adjunct in the presence of an exogenously supplied enzyme composition comprising a tannin uninhibited enzyme to provide the Brewer’ wort.

2. A method according to claim 1 wherein the tannin uninhibited enzyme is selected from the group consisting of a bacterial raw starch degrading α-amylase, glucoamylase, pullulanase, fungal alpha-amylase and a maltogenic alpha-amylase.

3. A method according to claim 2 wherein the grist comprises sorghum.

4. A method according to claim 3 wherein the grist comprises at least 10% sorghum.

5. A method according to claim 4 wherein the grist comprises at least 20% sorghum.

6. A method according to claim 5 wherein the grist comprises at least 30% sorghum.

7. A method according to claim 6 wherein the grist comprises at least 40% sorghum.

8. A method according to claim 7 wherein the grist comprises at least 50% sorghum.

9. A method according to claim 8 wherein the grist comprises at least 60% sorghum.

10. A method according to claim 9 wherein the grist comprises at least 70% sorghum.

11. A method according to claim 10 wherein the grist comprises at least 80% sorghum.

12. A method according to claim 11 wherein the grist comprises at least 90% sorghum.

13. A method according to claim 12 wherein the grist comprises 100% sorghum.

14. A method according to any of preceding claims wherein the grist further comprises corn, cassava, barley, wheat, rye, millet or rice.

15. A method according to any of claims 1 to 14 wherein the grist comprises at least 10 µM CAE/g of grist.

16. A method according to claim 15 wherein the grist comprises at least 20 µM CAE/g of grist.

17. A method according to claim 16 wherein the grist comprises at least 30 µM CAE/g of grist.

18. A method according to claim 17 wherein the grist comprises at least 40 µM CAE/g of grist.

19. A method according to claim 18 wherein the grist comprises at least 50 µM CAE/g of grist.

20. A method according to claim 19 wherein the grist comprises at least 60 µM CAE/g of grist.

21. A method according to claim 20 wherein the grist comprises at least 70 µM CAE/g of grist.

22. A method according to claim 21 wherein the grist comprises at least 80 µM CAE/g of grist.

23. A method according to claim 22 wherein the grist comprises at least 90 µM CAE/g of grist.

24. A method according to claim 23 wherein the grist comprises at least 100 µM CAE/g of grist.

25. A method according to claim 24 wherein the grist comprises at least 110 µM CAE/g of grist.

26. A method according to claim 25 wherein the grist comprises at least 120 µM CAE/g of grist.

27. A method according to claim 26 wherein the grist comprises at least 130 µM CAE/g of grist.

28. A method according to claim 27 wherein the grist comprises at least 140 µM CAE/g of grist.

29. A method according to claim 28 wherein the grist comprises at least 150 µM CAE/g of grist.

30. A method according to any of the preceding claims wherein the tannin uninhibited enzyme is a raw starch degrading α-amylase wherein said α-amylase is a glucohydrolase of class GH13.

31. A method according to claim 30 wherein the raw starch degrading α-amylase is derived from Cytophaga sp.

32. A method according to claim 31 wherein the raw starch degrading α-amylase has at least 60% sequence identity to SEQ ID NO:2 or an amylase active fragment thereof.

33. A method according to claim 32 wherein the raw starch degrading α-amylase has at least 65% sequence identity to SEQ ID NO:2 or an amylase active fragment thereof.

34. A method according to claim 33 wherein the raw starch degrading α-amylase has at least 70% sequence identity to SEQ ID NO:2 or an amylase active fragment thereof.

35. A method according to claim 34 wherein the raw starch degrading α-amylase has at least 75% sequence identity to SEQ ID NO:2 or an amylase active fragment thereof.

36. A method according to claim 35 wherein the raw starch degrading α-amylase has at least 80% sequence identity to SEQ ID NO:2 or an amylase active fragment thereof.

37. A method according to claim 36 wherein the raw starch degrading α-amylase has at least 85% sequence identity to SEQ ID NO:2 or an amylase active fragment thereof.

38. A method according to claim 37 wherein the raw starch degrading α-amylase has at least 90% sequence identity to SEQ ID NO:2 or an amylase active fragment thereof.

39. A method according to claim 38 wherein the raw starch degrading α-amylase has at least 95% sequence identity to SEQ ID NO:2 or an amylase active fragment thereof.

40. A method according to claim 39 wherein the raw starch degrading α-amylase has at least 98% sequence identity to SEQ ID NO:2 or an amylase active fragment thereof.

41. A method according to claim 40 wherein the raw starch degrading α-amylase has at least 99% sequence identity to SEQ ID NO:2 or an amylase active fragment thereof.

42. A method according to claim 41 wherein the raw starch degrading α-amylase has a sequence as set forth in SEQ ID NO:2 or an amylase active fragment thereof.

43. A method according to any of claims 30 to 42 wherein the exogenously supplied enzyme composition further comprises one or more of a protease, a fungal α-amylase, a maltogenic α-amylase and a lipase.

44. A method for determining whether an enzyme is inhibited by tannin comprising incubating the enzyme in the presence of tannin and detecting cross-linking of said enzyme to tannin.

45. A method according to claim 44 wherein the tannin comprises catechin.

46. A method according to any of claims 44 and 45 wherein the enzyme is a brewing enzyme.

47. A method according to claim 46 wherein the enzyme is selected from the group consiting of a raw starch degrading α-amylase, a protease, a fungal α-amylase, a maltogenic α-amylase and a lipase.

48. A method according to claim 47 wherein the enzyme is a raw starch degrading α-amylase.

49. A method according to claim 48 wherein said α-amylase is a glucohydrolase of class GH13.

50. A method according to claim 49 wherein the raw starch degrading α-amylase is derived from Cytophaga sp.

51. A method according to claim 50 wherein the raw starch degrading α-amylase has at least 60% sequence identity to SEQ ID NO:2 or an amylase active fragment thereof.

52. A method according to claim 51 wherein the raw starch degrading α-amylase has at least 65% sequence identity to SEQ ID NO:2 or an amylase active fragment thereof.

53. A method according to claim 52 wherein the raw starch degrading α-amylase has at least 70% sequence identity to SEQ ID NO:2 or an amylase active fragment thereof.

54. A method according to claim 53 wherein the raw starch degrading α-amylase has at least 75% sequence identity to SEQ ID NO:2 or an amylase active fragment thereof.

55. A method according to claim 54 wherein the raw starch degrading α-amylase has at least 80% sequence identity to SEQ ID NO:2 or an amylase active fragment thereof.

56. A method according to claim 55 wherein the raw starch degrading α-amylase has at least 85% sequence identity to SEQ ID NO:2 or an amylase active fragment thereof.

57. A method according to claim 56 wherein the raw starch degrading α-amylase has at least 90% sequence identity to SEQ ID NO:2 or an amylase active fragment thereof.

58. A method according to claim 57 wherein the raw starch degrading α-amylase has at least 95% sequence identity to SEQ ID NO:2 or an amylase active fragment thereof.

59. A method according to claim 58 wherein the raw starch degrading α-amylase has at least 98% sequence identity to SEQ ID NO:2 or an amylase active fragment thereof.

60. A method according to claim 59 wherein the raw starch degrading α-amylase has at least 99% sequence identity to SEQ ID NO:2 or an amylase active fragment thereof.

61. A method according to claim 60 wherein the raw starch degrading α-amylase has a sequence as set forth in SEQ ID NO:2 or an amylase active fragment thereof.

62. A method according to any of claims 44 to 61 wherein said step of detecting cross-linking comprises measuring turbidity.

63. A method according to any of claims 1 to 43 further comprising fermenting the Brewer’s wort to obtain an alcoholic beverage.

64. A method according to claim 63 wherein the alcoholic beverage is a beer.

65. A wort produced by the method of any of claims 1 to 44.

66. A beer produced by the method of claim 64.
